(19) United States
(12) Patent Application Publication
(10) Pub. No.: US 2004/0054160 A1
(43) Pub. Date: Mar. 18, 2004

(54) Nucleic-acid ink compositions for arraying onto a solid support

(76) Inventor: Santona Pal, Painted Post, NY (US)

Correspondence Address: CORNING INCORPORATED, SP-TI-3-1, CORNING, NY, 14831

(21) Appl. No.: 10/244,898

(22) Filed: Sep. 16, 2002

(51) Int. Cl. C12Q 1/68; C07H 21/04

(52) U.S. Cl. 536/24.3; 435/6

(57) ABSTRACT

A medium or ink solution containing nucleic acid is provided for depositing onto a solid support in the manufacture of biological arrays. The medium has a composition that comprises: about 30% to about 80% by volume of an organic solution comprising dimethylsulfoxide (DMSO), ethylene glycol (EG), formamide, or a combination thereof; a buffer with a pH value of about 3.5-9.5; water; and nucleic acid, wherein the nucleic acid denatures to provide for more favorable hybridization. The buffer can be made from a solution that may contain acetate, citrate, citrate-phosphate, maleate, or succinate. The medium permits long-term storage of nucleic acids in solution without excessive degradation, which is a phenomenon associated with many conventional ink solutions.

### RELATED APPLICATION

U.S. patent application Ser. No. 09/859,160, filed on May 16, 2001, in the names of Melanie C. Koroulis and Santona Pal.

### FIELD OF THE INVENTION

The present invention relates to the fabrication of high-density nucleic acid arrays for use in biological assays. In particular, the invention pertains to the formulation of a solution containing the nucleic acid, also referred to as an “ink.”

### BACKGROUND

Hybridization is widely used to test for the presence of a nucleic acid sequence that is complementary to a probe moiety. In many cases, this provides a simple, fast, and inexpensive alternative to conventional sequencing methods. Hybridization does not require nucleic acid cloning and purification, carrying out base-specific reactions, or tedious electrophoretic separations. Hybridization of oligonucleotide probes has been successfully used for various purposes, such as analysis of genetic polymorphisms, diagnosis of genetic diseases, cancer diagnostics, detection of viral and microbial pathogens, screening of clones, genome mapping and ordering of fragment libraries.

In heterogeneous assays, nucleic acid arrays may comprise a number of individual oligonucleotide species tethered to the surface of a solid support in a regular pattern, each species in a different area, so that the location of each oligonucleotide is known. An array can contain a chosen collection of oligonucleotides (e.g., probes specific for all known clinically important pathogens or specific for all known clinically important pathogens or specific for all known sequence markers of genetic diseases). Such an array can satisfy the needs of a diagnostic laboratory. Alternatively, an array can contain all possible oligonucleotides of a given length n. Hybridization of a nucleic acid with such a comprehensive array results in a list of all its constituent n-mers, which may be used for a number of assays. Examples include: for unambiguous gene identification (e.g., in forensic studies), for determination of unknown gene variants and mutations (including the sequencing of related genomes once the sequence of one of them is known), for overlapping clones, and for checking sequences determined by conventional methods. Finally, surveying the n-mers by hybridization to a comprehensive array can provide sufficient information to determine the sequence of a totally unknown nucleic acid.

An oligonucleotide array can be prepared by synthesizing all the oligonucleotides, in parallel, directly on the support, employing the methods of solid-phase chemical synthesis in combination with site-directing masks, such as described in U.S. Pat. No. 5,510,270. Four masks with non-overlapping windows and four coupling reactions are required to increase the length of tethered oligonucleotides by one. In each subsequent round of synthesis, a different set of four masks is used, and this determines the unique sequence of the oligonucleotides synthesized in each particular area. Using an efficient photolithographic technique, miniature arrays containing as many as 10⁵ individual oligonucleotides per cm² of area have been demonstrated.

Another technique for creating oligonucleotide arrays involves precise drop deposition using a piezoelectric pump, such as described in U.S. Pat. No. 5,474,796. A piezoelectric pump delivers minute volumes of liquid to a substrate surface. The pump design is very similar to the pumps used in ink jet printing. This picopump is capable of delivering a 50 micron-diameter (˜65 picoliter) droplets at up to 3000 Hz and can accurately hit a 250 micron target. As illustration, the pump unit may be assembled with five nozzles array heads, one for each of the four nucleotides and a fifth for delivering, activating agent for coupling. The pump unit remains stationary while droplets are fired downward at a moving array plate. When energized, a microdroplet is ejected from the pump and deposited on the array plate at a functionalized binding site. Different oligonucleotides are synthesized at each individual binding site based on the microdrop deposition sequence.

A popular method for creating high-density arrays uses pins, which are dipped into solutions of biological sample fluids and then touched to a surface. The nucleic acid (e.g., oligonucleotides or DNA) is typically solubilized in an aqueous medium (sometimes referred to as a “printing ink” or “ink”) that contains salts, which are used as components of buffers that are compatible with biological macromolecules. A 3×SSC (450 mM sodium chloride and 45 mM sodium citrate) is a standard concentration for printing inks. See, e.g., U.S. Pat. No. 5,807,522 (Example 1).

Use of SSC-containing inks, however, can be problematic. The first problem encountered in manufacturing DNA arrays using a 3×SSC ink is that the rate of evaporation of the aqueous medium is very high compared to the time required to print multiple slides. This is a major obstacle to scaling up the manufacturing process. Additionally, the present inventors have observed that not only is the 3×SSC ink incapable of printing the required number of slides, but also the quality and performance of arrays printed vary due to the evaporation of aqueous medium which results in a rapidly changing concentration of DNA.

Hence, a need exists for an ink composition for printing high-density arrays (HDAs) of nucleic acids that overcome the disadvantages in the art.

### SUMMARY OF THE INVENTION

The present invention provides, in part, an ink or medium for suspending a solution of nucleic acid, which may be deposited on a solid support. The medium has a composition that comprises about 30% to about 80% by volume of an organic solution comprising dimethylsulfoxide (DMSO), ethylene glycol (EG), formamide, or a combination thereof, a buffer with a pH value of about 3.5-9.5, water, and nucleic acid, wherein the nucleic acid denatures to provide for more favorable hybridization. The buffer is made from a solution that may include acetate, citrate, citrate-phosphate, maleate, or succinate. With increasing concentrations of DMSO in the ink, the pH value of the whole system also increases. Thus, buffered solutions with low pH values are required to compensate for the strong alkaline nature of DMSO. The medium possess a degree of stability that permits long-term storage of nucleic acids in solution without excessive degradation, which is a phenomenon associated with many conventional ink solutions. When used to print high-density arrays (HDAs), the present medium facilitates fabrication at high volumes over an extended period of time, such as over at least 20-30 days. Moreover, the medium enables superior adhesion to a functionalized substrate surface, as well as enhanced hybridization efficiency of the printed nucleic acid. It is believed that the present ink solutions can induce nucleic acids to show increased fluorescent signal when hybridized.

Other reagents can be incorporated as part of the ink composition, including those that would change the viscosity of the ink for enhancing wettability for certain printing conditions, for example, glycerol, histone proteins, etc. The inks may also contain small amounts of polycationic agents such as poly-lysine, spermine etc.

To facilitate denaturation of the nucleic acid, the nucleic acid may be suspended in the composition for at least 1 day, preferably longer (e.g., about 5-10 or 15 days), prior to printing.

In another aspect, the present invention pertains to a method for making a biological array. The method comprises contacting or otherwise depositing on a solid support an ink solution according to the present invention. Depositing step further comprises immersing a tip of a pin into the medium; removing said tip from the medium with the medium adhered to the pin tip; and transferring the ink solution to the solid support. The depositing step can be repeated a plurality of times to provide one or more arrays of nucleic acid. This can be accomplished, for example, by using a typographic pin array.

Additional features and advantages of the present ink solution will be explained in the following detailed description. It is understood that both the foregoing general description and the following detailed description and examples are merely representative of the invention, and are intended to provide an overview for understanding the invention as claimed.

### BRIEF DESCRIPTION OF THE DRAWINGS

FIGS. 1A and 1B illustrate the denaturation of nucleic acid samples in DMSO:SSC inks. FIG. 1A depicts an agarose gel showing the conformational state of DNA exposed to inks differing in the concentration of DMSO or EG at about 4 days after bio-formating. FIG. 1B shows in comparison the change in the conformational state of the same DNA samples at about 21 days after bioformating.

FIG. 2 is a schematic that depicts the conformational states of double-stranded DNA in denaturing solvents over time, as based on observations of electrophoretic mobility of the DNA.

FIGS. 3A and 3B show an agarose gel showing the conformational state of DNA that is exposed to DMSO based inks that do not contain any salts. 70% DMSO is effective in completely denaturing the 1.5 kB DNA fragment immediately after bioformating. FIG. 3B shows that the denaturing potential increases with time. Complete denaturation is achieved by 60% DMSO after 15 days of bioformatting.

FIGS. 4A and 4B show false-color images of hybridized arrays printed with eight different ink buffer systems, each at three different pH values.

FIGS. 5A-5F show false color images of cDNA hybridization on a microarray printed on CMT-GAPS slides with 22 yeast ORFs and a 1.5 kB fragment of DNA in six different inks.

FIGS. 6A and 6B shows a comparison of respective hybridization signals from four different ink compositions printed on an array.

FIG. 7A shows a false-color image of cDNA hybridization on a microarray using three ink compositions: Composition 1 is a non-buffered ink; Composition 2 is 50% DMSO: citrate at pH ˜5.5; and, Composition 3 is a mixed ink 50% DMSO: 30% EG: citrate at pH ˜5.5.

FIG. 7B depicts the differences in the average hybridization signal from the Cy3 and Cy5 channels for the genes due to the inks tested.

### DETAILED DESCRIPTION OF THE INVENTION

For high-volume manufacture of high-density arrays (HDAs), it is imperative that nucleic acid (e.g., oligonucleotides, single or double stranded DNA, or RNA) remains suspended in solution throughout the course of the manufacturing run and that this stability is maintained without compromising the hybridization efficiency. The desired life of nucleic acid formatted in the ink is between about 4 months and one year. The present invention provides ink compositions that can meet these goals. By refining chemical characteristics of ink solutions, the present invention advances beyond previous research and has achieved certain surprising results. The present invention improves stability and overcomes the problems and disadvantages associated with previous ink compositions, such as described in U.S. patent application Ser. No. 09/859,160, which is incorporated herein by reference. Relative to ink solutions which contain DMSO:SSC, the ink compositions of the present invention not only remedy the problem of evaporation, in part, by reducing the concentration of water, but also can control against excessive denaturation and provide better stability for strands of nucleic acid suspended in the ink.

Aqueous evaporation is a major obstacle to large volume manufacture of printed microarrays when using nucleic acid ink solutions that are largely aqueous and typically contain saline sodium citrate (SSC), such as of 3×SSC (450 mM sodium chloride and 45 mM sodium citrate) or greater concentration. In most nucleic-acid printing processes, an ink solution is frequently exposed to atmospheric conditions, which promotes desirable evaporation of the solvents in the ink. An undesired consequence, however, is evaporation of water from the ink solution, which results in progressively concentrated levels of organic components. For a commercial pin-printing process, such evaporation can not be tolerated. Since evaporation results typically in a constantly changing ink composition with an enriched DMSO concentration, commercially produced arrays would suffer from inconsistent quality.

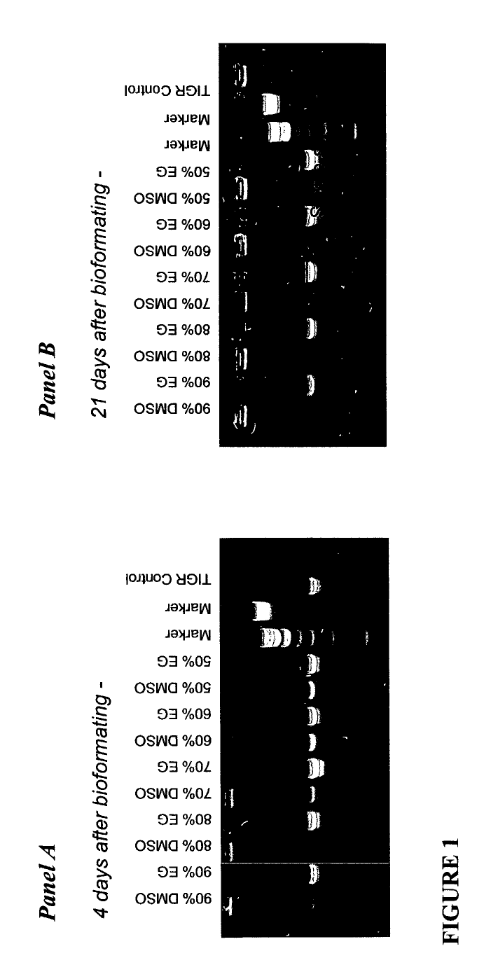

Over time, the changes in DMSO content and associated pH results in progressive denaturation of nucleic acids. For instance, FIGS. 1A and 1B shows denatured nucleic acid in DSMO:SSC-based inks. The salt concentration in these inks is kept constant at 0.25×SSC to monitor the effect of the organic component only. FIG. 1A is a picture of an agarose gel depicting the extent that 1.5 kB DNA fragments, which have been solvated in a selection of inks containing increasing concentrations (50%-90% v/v.) of DMSO or ethylene glycol (EG), is denatured after about 4 days. (The process is also referred to as bio-formating.) FIG. 1B shows the state of the same DNA samples after about 21 days of exposure to the inks. The appearance of new, faster-moving bands in the DMSO-based inks, in contrast to the EG-based inks, with electrophoretic mobility like single stranded DNA, suggests that effective denaturation takes place at high concentrations of DMSO. (Ts'o, P. O. P. et al., Tetrahedron, 1961, 13, 198; Zimmerman, E. et al. Biochemische Zeitschrift, 1966, 344, 386.)

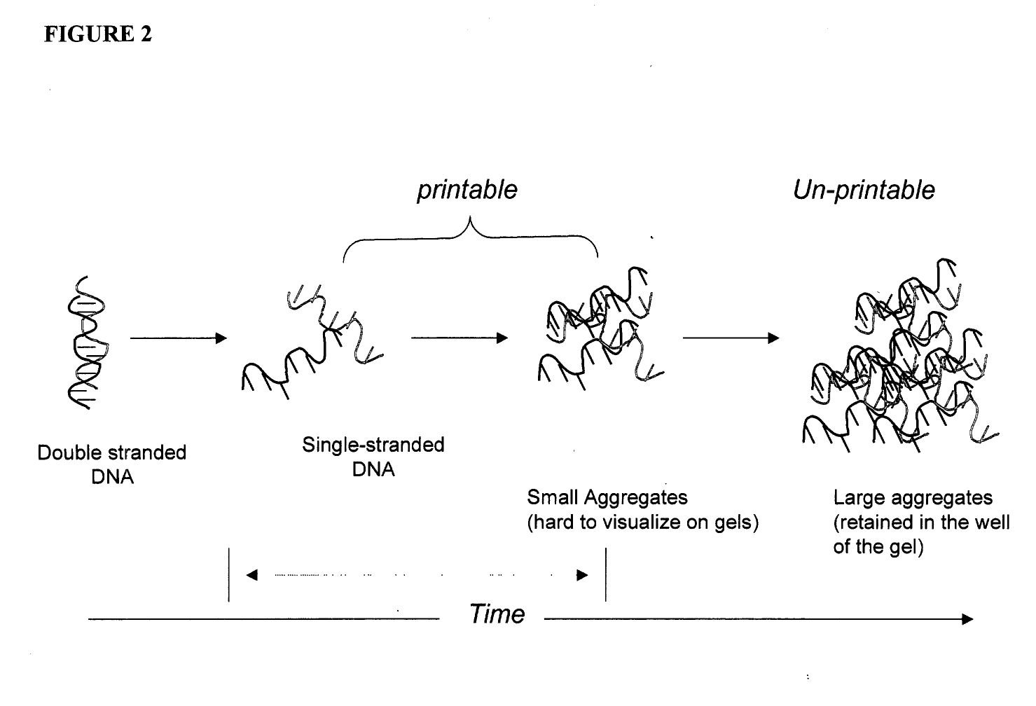

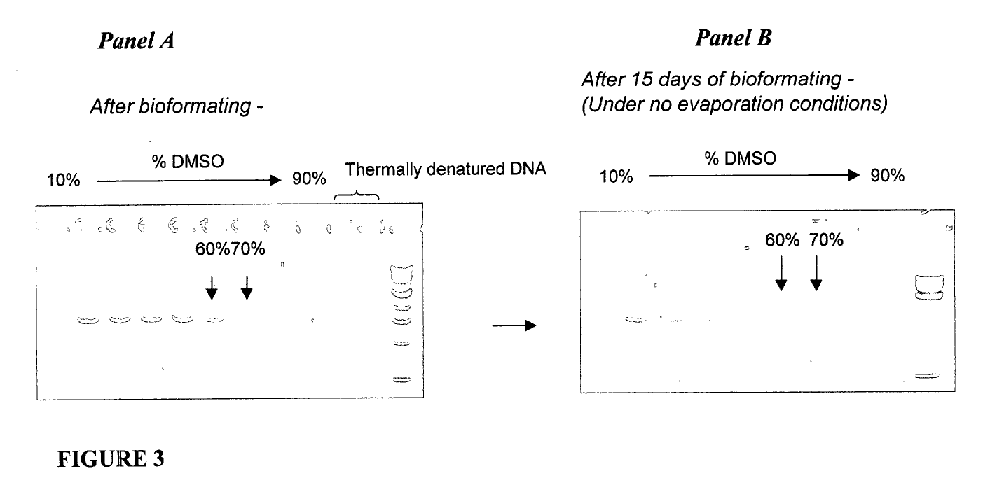

Although denaturation of nucleic acids is beneficial for an enhanced hybridization response, excessively denatured species are prone to form large aggregates, which have a tendency to precipitate out of solution. DNA aggregates are retained in the wells of the gel because of their inability to sieve through the gel matrix (FIG. 1B). As FIG. 2 depicts schematically, denatured species aggregate as time progresses. Hence, exposure of the nucleic acids to increasing concentrations of DMSO can degrade the nucleic acids over relatively long periods of time such as needed in high capacity, commercial printing operations, and consequently reduces the reproducibility of printed arrays. Further, since salts have reduced solubility in solutions with high DMSO concentrations, the salts in the ink can influence the aggregation and precipitation of nucleic acids, even though present in relatively low amounts. Moreover, as data from FIGS. 3A and 3B indicate, denaturation of DNA takes place in DMSO inks even in the absence of salts.

The most important effect of the salt is in influencing the wettability of components such as printing pins and coated slide surface. In the manufacture of arrays, it is desirable for a nucleic-acid ink to be able to wet thoroughly contact printing pins and to transfer completely from the pins to a functionalized surface of a substrate. In other words, the ink should adhere to the pins and adsorb to the surface in large amounts. Thus, one must make careful determination of pH and other parameters, including salt and organic solvent concentrations.

The present ink compositions overcome the problems associated with evaporation by, in part, reducing the concentration of water. Moreover, we have also discovered unexpectedly at least four other advantages of the present composition. First, the composition permits long-term storage of nucleic acid, which now enables sustained, continuous, high-volume array production. Before, short-term storage was a perennial problem in the art that went unsolved. Second, the composition produces a printable ink solution that provides superior adhesion, hybridization efficiency and response from nucleic acid species printed on binding substrates. For charged substrate surfaces, the relatively low salt concentration in the present ink compositions reduces ionic strength of the solution for better binding of nucleic acids to substrates. Third, a composition with a DMSO concentration of about 60% or greater by volume, results in augmented levels of denaturization, which even more unexpectedly, increases over time. The inventors also discovered, however, that DMSO at concentrations of over about 80% results in excessive denaturization, leading to aggregation of highly denaturized nucleic acid, which precipitate out of solution and cannot effectively hybridize in assay. Fourth, a combination of DMSO, low levels of salt, and controlled pH produces a preferred spot morphology when printed. This feature enables better contrast detection of printed spots. Traditionally, people thought that with a higher the salt concentration, one would achieve a better visual contrast. The inventors, however, have found that at relatively low concentrations, a favorable light scatter is also achievable. Salt, it is believed, crystallizes out of solution upon drying of the solvent components of the ink.

Hence, the present medium provides an optimal composition that reduces evaporation, increases stability of suspended nucleic acids, improves detection of printed spots. It is believed that the medium absorbs moisture from air to overcome a net loss of solvent due to evaporation of the water component. In addition, the composition controls the denaturation of nucleic acids in solution over time. The nucleic acids manifest conformations more favorable for hybridization between nucleic sequences in assay than achieved with conventional printing inks. All these attributes are desirable in a nucleic-acid ink solution.

According to the invention, the printing ink composition contains water, nucleic acid, about 30% or 40% to about 80% by volume of dimethylsulfoxide (DMSO), ethylene glycol (EG), formamide, or combinations thereof, and a buffer with a final pH value in the range of about 3.5 to about 9.5, made from a solution containing acetate, citrate, citrate-phosphate, or succinate. When the buffer contains acetic acid/acetate solution, the pH value is about 6 to about 8.5, preferably about 6.5 to about 7.5. When the buffer is a citric acid/citrate solution, the pH value is about 3.5 to about 7.5, preferably about 4 to about 6.5. When the buffer is a citric acid/citrate-phosphate solution, the pH value is about 6.0 to about 9, preferably about 7 to about 8.5. When the buffer is prepared with a succinic acid/OH/succinate solution, the pH value is about 3.5 to about 7, preferably about 4 to about 6.5. Maleate buffer systems at a pH value of about 5-5.5 may be used with mixed-solvent compositions containing either ethylene glycol or formamide, or used with DMSO at pH ˜8 to 8.5.

In some embodiments, when the composition contains about 40% to about 80% DMSO by volume, the buffer solution contains a final concentration of from about 0.1×(1.65 mM citric acid+0.85 mM sodium citrate) to about 0.8×(13.2 mM citric acid+6.8 mM sodium citrate). When the DMSO is about 40-70% by volume, the citrate buffer system contains a final concentration of about 0.1× to about 0.5×(8.25 mM citric acid+4.25 mM sodium citrate). Preferably, the solution contains about 40-60% DMSO by volume and the citrate buffer contains a final concentration of about 0.1× to about 0.4×(6.6 mM citric acid+3.4 mM sodium citrate). More preferably, the composition comprises about 50% DMSO by volume and citrate buffer at a final concentration of about 0.25×(4.125 mM citric acid+2.125 mM sodium citrate).

When the composition contains about 40% to about 80% DMSO by volume, acetic acid/acetate buffer solutions have a final concentration of about 0.1×(4.64 mM acetic acid +0.36 mM sodium acetate) to about 0.8×(37.12 mM acetic acid+2.88 mM sodium acetate). When the DMSO is about 40-70% by volume, the acetate buffer system contains a final concentration of about 0.1× to about 0.5×(23.2 mM acetic acid+1.8 mM sodium acetate). Preferably, the solution contains about 40-60% DMSO by volume and the acetate buffer contains a final concentration of about 0.1× to about 0.4×(18.56 mM acetic acid+1.44 mM sodium acetate). More preferably, the composition comprises about 50% DMSO by volume and acetate buffer at a final concentration of about 0.25×(11.6 mM acetic acid+0.9 mM sodium acetate).

When the composition contains about 40% to about 80% DMSO by volume, buffer solutions based on citric acid/citrate-phosphate, have a final concentration of about 0.1×(1.52 mM citric acid+1.93 mM sodium phosphate) to about 0.8×(12.16 mM citric acid+15.44 mM sodium phosphate). When the DMSO is about 40-70% by volume, the citric acid/citrate-phosphate buffer system contains a final concentration of about 0.1× to about 0.5×(7.6 mM citric acid+9.65 mM sodium phosphate). Preferably, the composition contains 40-60% DMSO by volume and the citric acid/citrate-phosphate buffer system contains a final concentration of about 0.1× to about 0.4×(4.8 mM citric acid+7.72 mM sodium phosphate). More preferably, the composition comprises about 50% DMSO by volume and the citric acid/citrate-phosphate buffer system contains a final concentration of about 0.25×(3.8 mM citric acid+4.825 mM sodium phosphate).

When the composition contains about 40% to about 80% DMSO by volume, buffer solutions based on succinic acid/sodium hydroxide, have a final concentration of about 0.1×(2.5 mM succinic acid+0.75 mM sodium hydroxide) to about 0.8×(20.0 mM succinic acid+6.0 mM sodium hydroxide). When the DMSO is about 40-70% by volume, the succinic acid/sodium hydroxide buffer system contains a final concentration of about 0.1× to about 0.5×(12.5 mM succinic acid+3.75 mM sodium hydroxide). Preferably, the solution contains about 40-60% DMSO by volume and the succinic acid/sodium hydroxide buffer system contains a final concentration of about 0.1× to about 0.4×(10 mM succinic acid+3 mM sodium hydroxide). More preferably, the composition comprises about 50% DMSO by volume and the succinic acid/sodium hydroxide buffer system contains a final concentration of about 0.25×(6.25 mM succinic acid+1.875 mM sodium hydroxide).

In other embodiments, the ink comprises a mixed organic solution of about 1% to about 50% or 55% by volume of ethylene glycol (EG) or formamide, either individually or together, or with DMSO. Ink compositions with mixed organic solutions, in addition to having superior hybridization response from nucleic acid species, also provides good nucleic acid stability beyond mere control of pH, which facilitates the manufacture of arrays at high volume over long printing runs.

Preferably, the ink composition comprises about 40% to about 80% DMSO by volume and citrate buffer in a final concentration from about 0.1× to about 0.8×, as specified above. More preferably, the composition comprises about 40% to about 75% DMSO by volume and about 1% to 50% EG by volume and citrate buffer in final concentration from about 0.25× to about 0.5×. Most preferably, the composition comprises about 50% DMSO by volume about 10% to 40% EG by volume and citrate buffer in a final concentration of about 0.25×. Other buffer systems, such as those aforementioned, of course, also may be employed. Formamide can be substituted for ethylene glycol in certain embodiments. In embodiments that include ethylene glycol and/or formamide, the organic solution preferably comprises about 5% to about 40% EG/formamide by volume. More preferably, the solution comprises about 10% to about 30% EG/formamide by volume. The tables in the examples that follow further detail the buffer concentrations and pH in the present compositions.

The ink composition may also include ethylene-diamine-tetra-acetic acid (EDTA) in a final concentration between 0 and about 4 mM, preferably 0.5 mM. Other agents can be incorporated as part of the ink composition, including those (e.g., glycerol, etc.) that can change the viscosity of the ink for enhancing wettability and desirable rheological properties to the composition for deposition with a probe tip or for certain printing conditions. The inks may contain low concentrations of multivalent, cationic, organic and inorganic molecules such as cobalt (III) hexa-amine, spermine, spermidine, poly-lysine, histone proteins, etc. The positive charge on these molecules cause condensation or self-association of the DNA fragments by bridging the negative charges on neighboring DNA fragments. Neutral polymers (e.g., dextran) in small amounts could also improve the retention properties of the nucleic acid in the medium to a printed substrate surface. These alternative non-cationic agents can potentially alleviate any complications arising out of poly-cationic condensing agents. Moreover, they are applicable to all HDA substrates, and are not necessarily limited to positively charged HDA substrates.

The ink composition enables long-term storage and preserves integrity of nucleic acid without instability by precipitation or aggregation of said nucleic acid. Consequently, the composition enables prolonged printing over at least 15-20 days.

The nucleic acid used in the ink composition and method of the present invention may include oligonucleotides, deoxyribonucleic acid (DNA) or ribonucleic acid (RNA). The nucleic acid may be single or double stranded. The nucleic acid may be, for example, a PCR product, PCR primer, or nucleic acid duplex. The nucleic acid is preferably a single or double stranded DNA or an oligonucleotide. The typical concentration of nucleic acid in the ink solution ranges from about 0.01 mg/ml to about 0.50 mg/ml, preferably about 0.25 mg/ml.

According to another aspect, the present invention provides a method for depositing a nucleic acid onto a solid support. The method includes the step of contacting or otherwise depositing on a solid support an ink solution according to the present invention. The depositing step further comprises immersing a tip of a pin into the ink solution; removing the tip from the ink solution with the ink adhered to the pin tip; and transferring the ink to the solid support. The depositing step can be repeated a plurality of times to provide one or more arrays of nucleic acid. This can be accomplished, for example, by using a typographic pin array. The depositing step may be carried out using an automated, robotic printer. Such robotic systems are available commercially from, for example, Intelligent Automation Systems (IAS), Cambridge, Mass.

The pin can be solid or hollow. The tips of solid pins are generally flat, and the diameter of the pins determines the volume of fluid that is transferred to the substrate. Solid pins having concave bottoms can also be used. To permit the printing of multiple arrays with a single sample loading, hollow pins that hold larger sample volumes than solid pins and therefore allow more than one array to be printed from a single loading can be used. Hollow pins include printing capillaries, tweezers and split pins. An example of a preferred split pen is a micro-spotting pin that TeleChem International (Sunnyvale, Calif.) has developed.

A typographical pin array having a matrix of pins aligned such that each pin from the matrix fits into a corresponding source well, e.g., a well from a microtiter plate, is preferably used to form HDAs. The pin array may also be used in conjunction with a redrawn capillary-imaging reservoir. See, International Patent Application WO 99/55460, incorporated herein by reference.

According to the method, any solid support may be employed, so long as it is capable of retaining the printed nucleic acid. The solid support preferably has a planar surface upon which the nucleic acid is deposited. The solid support is generally a membrane or glass substrate. For instance, the solid support is a two-dimensional solid glass surface, such as commercially available glass microscope slides (3″×1″) made of soda lime, or other glass compositions. Preferably, the substrate is made of either a boroaluminosilicate or a borosilicate glass (e.g., U.S. patent application Ser. No. 09/245,142). Other supports may include three-dimensional porous glass surfaces (e.g., Vycor™ by Corning Inc; U.S. patent application Ser. No. 10/101,144) or porous glass substrates made by tape-cast or sol-gel processes from Pyrex™ glass frit (e.g., U.S. patent application Ser. No. 10/101,135). It is preferred that glass substrates have a surface that is functionalized or coated to facilitate the adhesion of the nucleic acid. For instance, the surface may comprise a variety of reactive polar moieties, which may include: amino, hydroxyl, or alkyl-thiol groups, acrylic acid, esters, anhydrides (e.g., styrene-co-maleic anhydride (SMA copolymer)), aldehyde, epoxide or other protected precursors capable of generating reactive functional groups. A surface-coating, aminating agent is preferred, such as comprising polylysine or aminoalkylsilanes, such as gamma-aminopropylsilane (GAPS) (e.g., γ-aminopropyl trimethoxysilane, N-(beta-aminoethyl)-γ-aminopropyl trimethoxysilane, N-(beta-aminoethyl)-γ-aminopropyl triethoxysilane or N′-(beta-aminoethyl)-γ-aminopropyl methoxysilane).

In post printing processing, subjecting the slide to hot boiling water can further denature the nucleic acid (DNA) printed in DMSO/ethylene-gycol/formamide-based ink compositions (denaturing solvents). Enhanced signals obtained from the subsequent thermal denaturation of the printed slides suggest improving hybridization efficiency.

The arrays produced in accordance with the methods of the present invention may be interrogated using labeled targets (e.g., oligonucleotides, nucleic acid fragments such as cDNA and cRNA, PCR products, etc.). The targets may be labeled with fluorophores such as the Cy3, Cy5, or Alexa dyes, etc., or with other haptens such as biotin, digoxogenin. The methods for biotinylating nucleic acids are familiar and described by Pierce (Avidin-Biotin Chemistry: A Handbook. Pierce Chemical Company, 1992, Rockford, Ill.).

Alternatively, to detect the hybridization event (i.e., the presence of the biotin), the solid support may be incubated with streptavidin/horseradish peroxidase conjugate. Such enzyme conjugates are commercially available from, for example, Vector Laboratories (Burlingham, Calif.). The streptavidin binds with high affinity to the biotin molecule bringing the horseradish peroxidase into proximity to the hybridized probe. Unbound streptavidin/horseradish peroxidase conjugate is washed away in a simple washing step. The presence of horseradish peroxidase enzyme is then detected using a precipitating substrate in the presence of peroxide and the appropriate buffers. It is also possible to use chemiluminescent substrates for alkaline phosphatase or horseradish peroxidase (HRP), or fluorescence substrates for HRP or alkaline phosphatase. Examples include the diox substrates for alkaline phosphatase available from Perkin Elmer or Attophos HRP substrate from JBL Scientific (San Luis Obispo, Calif.).

Method for fabrication and use of high-density nucleic acid arrays are set forth in Microarray Biochip Technology, M. Schena, ed. Eaton Publishing, Natick, Mass. (2000). The patents and other documents cited throughout the present specification are incorporated herein by reference.

The examples in the following section further illustrate and describe the advantages and qualities of the present invention.

### EXAMPLES

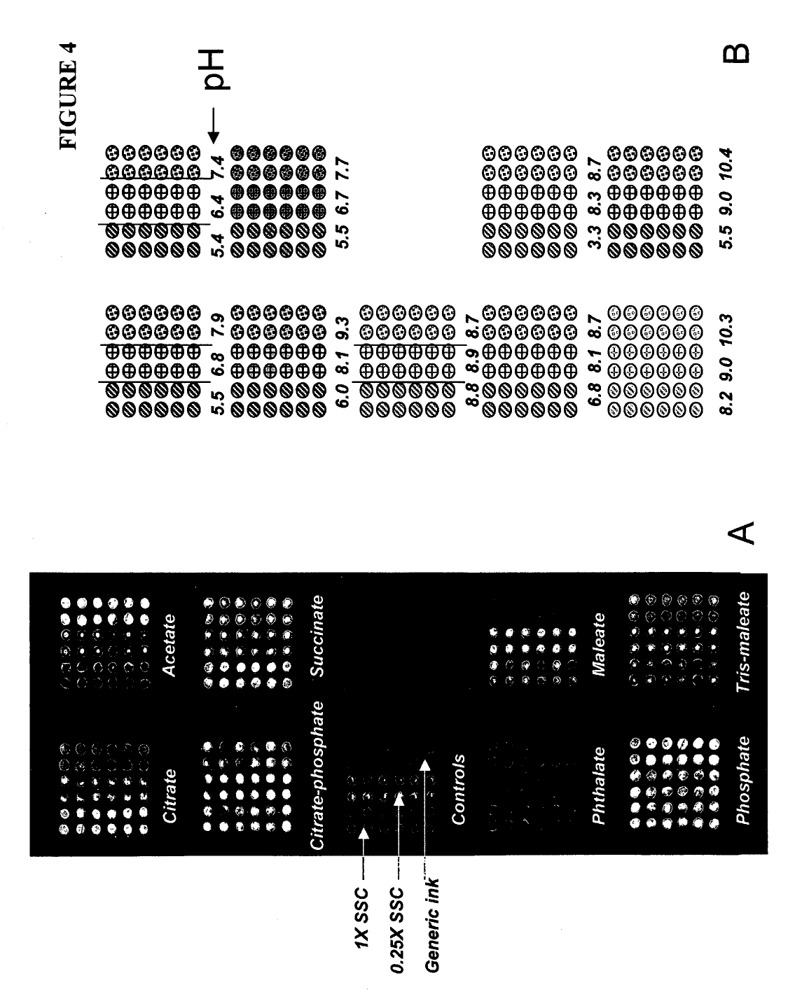

In a series of studies, using solvent solutions of 40%, 50% 70%, and 80% by volume of DMSO, ethylene glycol, or formamide, we prepared eight (8) different buffer systems, each at three (3) different pH values. FIG. 4A shows false color images of the respective arrays printed using ink solutions made with the eight buffer systems. Each ink solution contains 50% DMSO. Adjusting the buffer composition modifies the pH value of each ink solution. A 1.5 kB fragment of DNA is printed in each of the inks specified in panel A of the figure, and hybridized with Cy3-labeled complimentary DNA. For comparative control, in the center is an array printed, respectively from left to right, with two columns each of a 1×SSC-containing ink, a 0.25 SSC-containing ink, and generic standard DMSO-based ink. Each ink solution was screened for salt content, stability of bioformated nucleic acid (DNA), and hybridization response from the printed nucleic acid. From these studies, the ink compositions summarized in Table 1 are more stable than currently used DMSO:SSC inks and give either comparable or better hybridization responses.

TABLE 1

| | | |
|---|---|---|
| Ex. 1. | 50% DMSO: citrate (0.25× = 4.125 mM citric acid, | pH ˜5.5 |
| | 2.125 mM sodium citrate) | |
| Ex. 2. | 50% DMSO: citrate-phosphate (0.25× = 3.8 mM citric | pH ˜6.0 |
| | acid, 4.825 mM dibasic sodium phosphate) | |
| Ex. 3. | 50% DMSO: succinate (0.25× = 6.25 mM succinic | pH ˜5.5 |
| | acid, 1.875 mM NaOH) | |
| Ex. 4. | 50% DMSO: acetate (0.25× = 11.6 mM acetic acid, | pH ˜5.4 |
| | 0.9 mM sodium acetate) | |

As can be seen in both FIGS. 4A and 4B, the pH of the buffer system has a significant impact on the hybridization performance of a microarray printed using the present ink compositions. Hybridization using ink compositions containing citrate, citrate-phosphate, acetate or succinate performed better than the ink systems containing pthalate, phosphate, maleate, or tris-maleate, as well as the controls. Although the hybridization performance of the phosphate containing ink appears to be comparable with that of citrate or citrate-phosphate inks, phosphate salts are prone to precipitate in a medium containing DMSO solvent. Hence, a buffer composition of phosphate alone is not preferred.

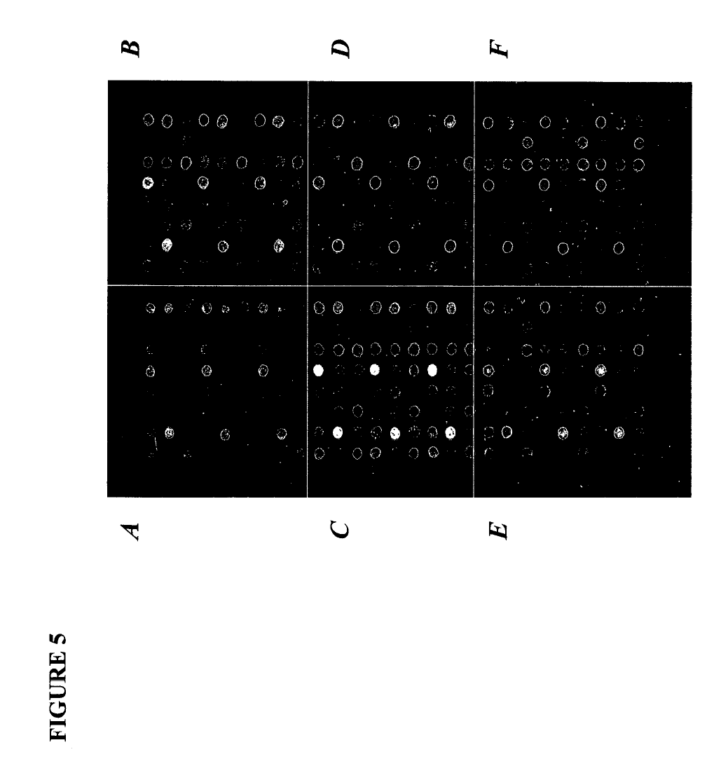

FIGS. 5A-5F show, in false color, a DMSO:citrate based ink, according to the present invention, compared with other printing ink solutions. On a glass slide coated with γ-aminopropylsilane (GAPS), 22 yeast ORFs and, as a control, a Cy5-labeled 1.5 kB fragment of pBR DNA are printed in six different inks. Each of the panels A-F is printed with a separate pin using a flexys robotic printer, and each piece of DNA is printed in triplicate. Panel A is printed using a 50% DMSO: 1×SSC-based ink; panel B using a 50% DMSO: 0.25×SSC-based ink; and, panel C using a 50% DMSO: citrate (0.25×, pH 5.5) ink. The inks employed in panels D and E did not contain DMSO. An aqueous 80% ethylene glycol-based ink and a 50% ethylene: 0.25×SSC-based ink, respectively, is used in panels D and E. Panel F is printed using a 50% formamide: 0.25× phosphate solution. Cy3 labeled yeast cDNA samples were hybridized to the printed microarray. The role of the inks in enhancing the signal intensities and, thereby, improving the sensitivity of the hybridization performance of the microarray is clearly depicted in the panels.

It was discovered that with respect to stability, all ethylene glycol (EG) and formamide based inks were superior, but generally exhibited lower hybridization signals than DMSO-based inks. Nonetheless, certain composition of ethylene glycol and formamide based inks could give comparable hybridization performance. Their compositions are detailed in Table 2.

TABLE 2

| | | |
|---|---|---|
| Ex. 1 | 50% Ethylene glycol: maleate (1.0× = 25 mM | pH |
| | sodium maleate, 3.6 mM sodium hydroxide) | ˜5-5.5 |
| Ex. 2 | 50% Ethylene glycol: acetate (1.0× = 46.3 mM acetic | pH |
| | acid, 3.7 mM sodium acetate) | ˜4-5.5 |
| Ex. 3 | 50% Formamide: maleate (1.0× = 25 mM sodium | pH |
| | maleate, 3.6 mM sodium hydroxide) | ˜5-5.5 |
| Ex. 4 | 80% EG: citrate (0.4× = 6.6 mM citric acid, 3.4 mM | pH ˜5.5 |
| | sodium citrate) | |
| Ex. 5 | 80% EG: succinate (0.4× = 10.0 mM succinic acid, | pH ˜5.5 |
| | 3.0 mM sodium hydroxide) | |
| Ex. 6 | 80% EG: citrate-phosphate (0.4× = 6.14 mM citric acid, | pH ˜5.4 |
| | 7.76 mM dibasic sodium phosphate); | |

The ethylene glycol and formamide based inks of Table 2 exhibited good hybridization signals and were stable at salt concentrations between 1.0× and 0.1× and under various pH conditions. More importantly, these inks maintain their stability, wherein nucleic acids remain suspended in compositions with up to 80% organic content. This is a valuable attribute since evaporation of water from the ink solution leads generally to a final composition that is rich in the organic component.

On one hand, DMSO in DMSO-based inks contributes favorably to the hybridization efficiency of the printed nucleic acids; however, DMSO cannot be used at high concentrations since it compromises the integrity of nucleic acids over an extended period of time. While on another hand, the ink compositions, which contain ethylene glycol and/or formamide are stable at high concentrations, are useful to reduce concentration losses due to aqueous evaporation since they lower the overall amount of water in solution. Inks that combine the favorable attributes of both the DMSO and EG based inks are potentially very beneficial.

Inks of mixed composition, such as listed in Table 3, containing both DMSO and ethylene glycol (EG)/formamide, are simultaneously stable and sufficiently denaturing of nucleic acids to satisfy both longevity for mass-production printing and requisite levels of hybridization efficiency.

TABLE 3

| | | |
|---|---|---|
| Ex. 1 | 40% DMSO, 10% EG/formamide: citrate (0.25× = | pH ˜5.5 |
| | 4.125 mM citric acid, 2.125 mM sodium citrate) | |
| Ex. 2 | 40% DMSO, 30% EG/formamide: citrate (0.25× = | pH ˜5.5 |
| | 4.125 mM citric acid, 2.125 mM sodium citrate) | |
| Ex. 3 | 40% DMSO, 40% EG/formamide: citrate (0.25× = | pH ˜5.5 |
| | 4.125 mM citric acid, 2.125 mM sodium citrate) | |
| Ex. 4 | 50% DMSO, 20% EG/formamide: citrate (0.25× = | pH ˜5.5 |
| | 4.125 mM citric acid, 2.125 mM sodium citrate) | |
| Ex. 5 | 50% DMSO, 30% EG/formamide: citrate (0.25× = | pH ˜5.5 |
| | 4.125 mM citric acid, 2.125 mM sodium citrate) | |

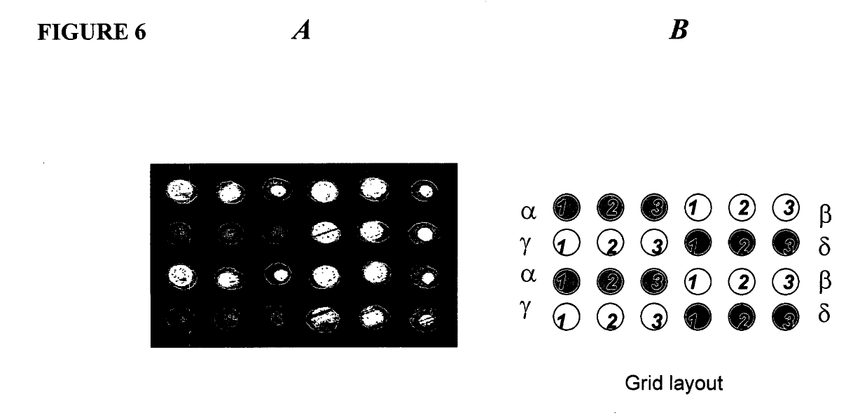

To reiterate, since the ink compositions that contained greater amounts of organic components were less affected by aqueous evaporation, these inks also suffered less from the associated deleterious problems. FIG. 6A (false color image) and FIG. 6B show a comparison of a hybridization done with Cy3 labeled 1.5 kB DNA on a DNA array printed with 1.5 kB DNA in four different ink compositions: α)—50% DMSO:SSC (0.25×); β)—50% DMSO:citrate (0.25×, pH ˜5.5); γ)—80% aqueous ethylene glycol; δ)—50% DMSO+30% ethylene glycol:citrate (0.25×, pH ˜5.5). The DNA was printed in different concentrations: 1) 0.25 mg/ml; 2) 0.125 mg/ml; 3) 0.06 mg/ml.

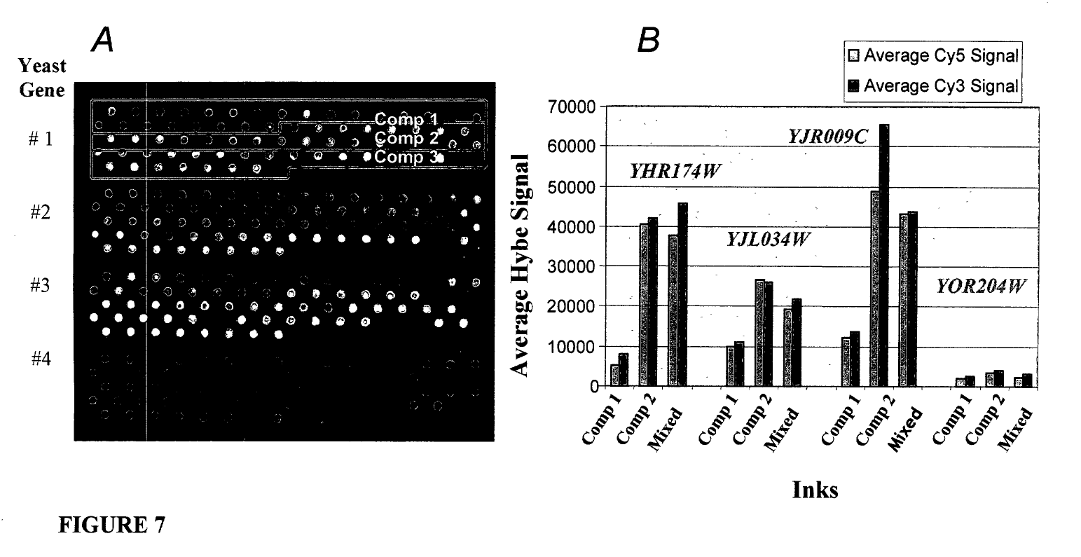

FIG. 7A depicts a false color image of yeast cDNA hybridization on a DNA microarray, consisting of 24 replicates each of 4 yeast genes, printed on GAPS-coated slides. Composition 1 (Comp 1) is a non-buffered ink. According to the present invention, composition 2 (Comp 2) is 50% DMSO:citrate at pH 5.5, and composition 3 (Mixed) is a mixed ink of 50% DMSO+30% ethylene glycol:citrate. FIG. 7B summarizes the differences in the average hybridization signal derived from the Cy3 and Cy5 channels for the genes due to the inks tested. As observed, the net retention and hybridization signal obtained with any of the inks above is dependent on the fragment sized and sequence of the DNA. Thus, signal may vary from gene to gene. The wettability of the ink depends on the physical properties of the materials with which the ink comes into contact, such as the surface energies of the printing surfaces. Or, in other words, the absolute signal form hybridization obtained with any ink is dependent on the materials of the pins and the slides.

Although the present invention has been described generally and in detail by way of examples and the figures, persons skilled in the art will understand that the invention is not limited necessarily to the embodiments specifically disclosed, but that modifications and variations can be made without departing from the spirit and scope of the invention. Therefore, unless changes otherwise depart from the scope of the invention as defined by the following claims, they should be construed as included herein.

We claim:

1. A medium for suspending a solution of nucleic acid, the medium having a composition comprising:
about 30% to about 80% by volume of an organic solution comprising dimethylsulfoxide (DMSO), ethylene glycol (EG), formamide, or a combination thereof,
a buffer with a pH value of about 3.5-9.5;
water; and
nucleic acid, wherein the nucleic acid denatures to provide for more favorable hybridization.

2. The medium according to claim 1, wherein said buffer is made from a solution that may include acetate, citrate, citrate-phosphate, maleate, or succinate.

3. The medium according to claim 2, wherein when said buffer includes acetate, the pH value is about 6 to about 8.5.

4. The medium according to claim 3, wherein the pH value is about 6.5 to about 7.5.

5. The medium according to claim 2, wherein when said buffer includes citrate, the pH value is about 3.5 to about 7.5.

6. The medium according to claim 5, wherein the pH value is about 4 to about 6.5.

7. The medium according to claim 2, wherein when said buffer includes citrate-phosphate, the pH value is about 6.0 to about 9.

8. The medium according to claim 7, wherein the pH value is about 7 to about 8.5.

9. The medium according to claim 2, wherein when said buffer includes succinate, the pH value is about 3.5 to about 7.

10. The medium according to claim 9, wherein the pH value is about 4 to about 6.5.

11. The medium according to claim 2, wherein when said buffer includes maleate, the pH value is about 5 to about 8.5.

12. The medium according to claim 11, wherein when said composition contains either ethylene glycol or formamide, said maleate buffer is at a pH value of about 5-5.5.

13. The medium according to claim 11, wherein when said composition contains DMSO, said maleate buffer is at a pH value of ˜8 to 8.5.

14. The medium according to claim 1, wherein said nucleic acid is at a concentration ranging from about 0.01 mg/ml to about 0.5 mg/ml.

15. The medium according to claim 1, wherein the nucleic acid is a double stranded DNA, RNA, or an oligonucleotide.

16. The medium according to claim 1, wherein said composition enables long-term storage and preserves integrity of nucleic acid without instability by precipitation or aggregation of said nucleic acid.

17. The medium according to claim 16, wherein said composition enables prolonged storage and printing over at least 15 days.

18. The medium according to claim 1, wherein said composition comprises about 40% to about 80% DMSO by volume, the buffer contains a final concentration of from about 0.1× to about 0.8× of citric acid+sodium citrate.

19. The medium according to claim 18, wherein said composition comprises about 50% DMSO by volume and citrate buffer at a final concentration of about 0.25× of citric acid+sodium citrate.

20. The medium according to claim 1, wherein said composition comprises about 40% to about 80% DMSO by volume and the buffer contains a final concentration of about 0.1× to about 0.8× of acetic acid+sodium acetate.

21. The medium according to claim 20, wherein said composition comprises about 50% DMSO by volume and acetate buffer at a final concentration of about 0.25× of acetic acid+sodium acetate.

22. The medium according to claim 1, wherein said composition comprises about 40% to about 80% DMSO by volume and the buffer contains a final concentration of about 0.1× to about 0.8× of citric acid+sodium phosphate.

23. The medium according to claim 22, wherein said composition comprises about 50% DMSO by volume and citric acid/citrate-phosphate buffer at a final concentration of about 0.25× of citric acid+sodium phosphate.

24. The medium according to claim 1, wherein said composition comprises about 40% to about 80% DMSO by volume and the buffer contains a final concentration of about 0.1× to about 0.8× of succinic acid+sodium hydroxide.

25. The medium according to claim 24, wherein said composition comprises about 50% DMSO by volume and succinic acid/sodium hydroxide buffer at a final concentration of about 0.25× of succinic acid+sodium hydroxide.

26. The medium according to claim 1, wherein said composition may include EDTA in a final concentration between 0 and about 4 mM.

27. The medium according to claim 26, wherein said composition includes EDTA in a final concentration of about 0.5 mM.

28. The medium according to claim 1, wherein said composition may include agents that can change the viscosity of the ink for enhancing wettability.

29. The method according to claim 28, wherein said composition may include either or both multivalent, cationic, organic and inorganic molecules, and/or neutral polymers.

30. The medium according to claim 29, wherein said cationic molecules include cobalt (III) hexa-amine, spermine, spermidine, poly-lysine, histones.

31. A medium for suspending a solution of nucleic acid, the medium having a composition comprising: a mixed organic solution of about 1% to about 55% by volume of ethylene glycol (EG) or formamide either individually, together, or with DMSO; a buffer with a pH value of about 3.5-9.5; water; and nucleic acid.

32. The medium according to claim 31, wherein said composition comprises about 40% to about 80% DMSO by volume and citrate buffer at a final concentration from about 0.1× to about 0.8×.

33. The medium according to claim 31, wherein said composition comprises about 40% to about 75% DMSO by volume and about 1% to 50% EG by volume and citrate buffer at final concentration from about 0.25× to about 0.5×.

34. The medium according to claim 31, wherein said composition comprises about 50% DMSO by volume about 10% to 40% EG by volume and citrate buffer at a final concentration of about 0.25×.

35. The medium according to claim 31, wherein said composition includes an organic solution comprising about 5% to about 40% EG/formamide by volume.

36. The medium according to claim 31, wherein said composition includes an organic solution comprising about 10% to about 30% EG/formamide by volume.

37. The medium according to claim 31, wherein said composition may include EDTA in a final concentration between 0 and about 4 mM.

38. A method for depositing a nucleic acid on a support, said method comprising:
a) providing a solution of nucleic acid comprising about 30% to about 80% by volume of dimethylsulfoxide (DMSO), ethylene glycol (EG), formamide, or a combination thereof; a buffer with a pH value of about 3.5-9.5; water; and a nucleic acid; and
b) depositing said solution on the support.

39. The method of claim 38, wherein the depositing step comprises immersing a tip of a pin into the solution of nucleic acid; removing said tip from said solution to provide solution adhered to said tip; and transferring said solution to the support.

40. The method of claim 38, wherein the depositing step is repeated a plurality of times to provide one or more arrays of nucleic acid.

41. The method according to claim 38, wherein said buffer is made from a solution that may include acetate, citrate, citrate-phosphate, maleate, or succinate.

42. The method according to claim 41, wherein when said buffer is an acetic acid system, the pH value is about 6 to about 8.5.

43. The method according to claim 42, wherein the pH value is about 6.5 to about 7.5.

44. The method according to claim 41, wherein when said buffer is a citric acid system, the pH value is about 3.5 to about 7.5.

45. The method according to claim 44, wherein the pH value is about 4 to about 6.5.

46. The method according to claim 41, wherein when said buffer is a citrate-phosphate system, the pH value is about 6.0 to about 9.

47. The method according to claim 46, wherein the pH value is about 7 to about 8.5.

48. The method according to claim 41, wherein when said buffer is a succinic acid system, the pH value is about 3.5 to about 7.

49. The method according to claim 48, wherein the pH value is about 4 to about 6.5.

50. The method according to claim 41, wherein when said buffer is a maleate system, the pH value is about 5 to about 8.5.

51. The method according to claim 50, wherein when said composition contains either ethylene glycol or formamide, said maleate buffer is at a pH value of about 5-5.5.

52. The method according to claim 50, wherein when said composition contains DMSO, said maleate buffer is at a pH value of ˜8 to 8.5.

53. The method according to claim 38, wherein said nucleic acid is at a concentration ranging from about 0.01 mg/ml to about 0.5 mg/ml.

54. The method according to claim 38, wherein the nucleic acid is a double stranded DNA, RNA, or an oligonucleotide.

55. The method according to claim 38, wherein said composition enables long-term storage and preserves integrity of nucleic acid without instability by precipitation or aggregation of said nucleic acid.

56. The method according to claim 38, wherein said composition enables prolonged storage and printing over at least 15 days.

57. The method according to claim 38, wherein said solution comprises about 40% to about 60% DMSO by volume and a buffer at a final concentration of from about 0.1× to about 0.4× of citric acid+sodium citrate.

58. The method according to claim 57, wherein the solution comprises about 50% DMSO by volume and the buffer at a final concentration of about 0.25× of citric acid+sodium citrate.

59. The method according to claim 38, wherein said solution comprises about 40% to about 60% DMSO by volume and a buffer at a final concentration of from about 0.1× to about 0.4× of acetic acid+sodium acetate.

60. The method according to claim 38, wherein said solution comprises about 40% to about 60% DMSO by volume and a buffer at a final concentration of from about 0.1× to about 0.4× citric acid+sodium phosphate.

61. The method according to claim 38, wherein said solution comprises about 40% to about 60% DMSO by volume and a buffer at a final concentration of from about 0.1× to about 0.4× succinic acid+sodium hydroxide.

62. The method according to claim 38, wherein the support has a planar surface capable of retaining the nucleic acid.

63. The method according to claim 62, wherein the support is either a membrane or a glass substrate.

64. The method according to claim 63, wherein said glass substrate is either a two-dimensional, solid glass surface or a three-dimensional, porous glass surface.

65. The method according to claim 62, wherein said glass substrate comprises a surface that is functionalized to facilitate adhesion of the nucleic acid.

66. The method according to claim 65, wherein said glass substrate is coated with anhydride functional groups.

67. The method according to claim 68, wherein said glass substrate is coated with a styrene-co-maleic anhydride (SMA) copolymer.

68. The method according to claim 65, wherein said glass substrate comprises an aminated surface.

69. The method according to claim 68, wherein said aminated surface is coated with an aminoalkylsilane.

70. The method according to claim 68, wherein said aminated surface is coated with an aminating agent comprising either y-aminopropylsilane or polylysine.

71. A method for depositing a nucleic acid on a solid support, said method comprising: depositing on the solid support a solution of nucleic acid comprising a mixed organic solution of about 1% to about 55% by volume of ethylene glycol (EG) or formamide either individually, together, or with DMSO; a buffer with a pH value of about 3.5-9.5; water; and nucleic acid.

72. The method according to claim 71, wherein said composition comprises about 40% to about 80% DMSO by volume and citrate buffer at a final concentration from about 0.1× to about 0.8×.

73. The medium according to claim 71, wherein said composition comprises about 40% to about 75% DMSO by volume and about 1% to 50% EG by volume and citrate buffer at final concentration from about 0.25× to about 0.5×.

74. The method according to claim 71, wherein said composition comprises about 50% DMSO by volume about 10% to 40% EG by volume and citrate buffer at a final concentration of about 0.25×.

75. The method according to claim 71, wherein said composition includes an organic solution comprising about 5% to about 40% EG/formamide by volume.

76. The method according to claim 71, wherein said composition includes an organic solution comprising about 10% to about 30% EG/formamide by volume.

77. The method according to claim 71, wherein the nucleic acid is at a concentration ranging from about 0.01 mg/ml to about 0.5 mg/ml.

78. The method according to claim 71, wherein the nucleic acid is DNA

79. The method according to claim 71, wherein the nucleic acid is an oligonucleotide.

80. The method according to claim 71, further comprising subjecting the nucleic acid on said solid support to thermal denaturation.
